(19) United States
(12) Patent Application Publication
(10) Pub. No.: US 2005/0099294 A1
(43) Pub. Date: May 12, 2005

(54) System for managing conditions

(76) Inventors: James T. Bogner, Indianapolis, IN (US); William G. Hopkins, Laguna Hills, CA (US); Robert Bull Brill, Newfield, NY (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/912,322

(22) Filed: Aug. 5, 2004

Related U.S. Application Data
(60) Provisional application No. 60/492,547, filed on Aug. 5, 2003.

(51) Int. Cl. G08B 23/00; A61B 5/00

(52) U.S. Cl. 340/540; 340/573.1; 340/573.5; 340/573.7; 340/584; 340/588; 600/300; 600/549; 600/481; 600/529

(57) ABSTRACT

A condition management system monitors a variety of biological and environmental conditions. Information collected with respect to conditions monitored are processed to create an information hierarchy that controls the operation of various alert devices. Important or critical conditions take precedent over less critical conditions both in terms of the nature of the alert and the processing order.

### CROSS-REFERENCE TO RELATED APPLICATION

The present application claims the benefit of U.S. Provisional Application Ser. No. 60/492,547 filed on Aug. 5, 2003, which is hereby incorporated by reference in its entirety.

### FIELD OF THE INVENTION

The present invention relates generally to methods and apparatus for monitoring and detecting conditions and more particularly to managing the information obtained from such condition monitoring and detecting.

### BACKGROUND OF THE INVENTION

It is becoming increasingly important to be able to accurately monitor the status of a variety of conditions for both people and objects, as well as being able to access and use the information collected in a variety of ways both local and distant from the source of the monitored condition. For example, it is estimated that one third to one half of all nursing home residents are incontinent. Incontinence problems are often neglected, unchecked or otherwise mismanaged by healthcare personnel, thereby causing discomfort and unpleasantness for the patients which often creates further health complications. As an example, many residents suffer from pressure sores (decubitus ulcers) that occur as a result of unrelieved pressure on the skin due to lack of movement. These decubitus ulcers are exacerbated by poorly managed incontinence problems, i.e., failure to change a patient's diaper or bedding when wet.

The problems in nursing homes and other managed care facilities are not limited to incontinence management. A state of crisis has been recognized in the U.S. among nursing homes, largely attributed to shortages in qualified caregivers. Common problems in care received by patients include, in addition to incontinence, malnutrition, dehydration, irregular and incorrect administration of medication, patients wandering outside of their rooms and abuse of patients by healthcare workers. The poor care received in these institutions is often most frustrating for the patients' loved ones, who often cannot regularly monitor the level of care their friend or family member is receiving.

Furthermore, the problems associated with monitoring individuals are not limited to nursing homes or health care facilities. They also extend to daycare facilities, nurseries, schools, and even individual homes. When the care of infants or children is involved, problems may arise ranging from a child who wanders unnoticed from their room to an infant who stops breathing while they are supposedly asleep.

Additionally, there is a growing need to monitor objects or locations as well as individuals. Improper storage and handling of food products may result in sickness or even death of people who eat such food. The fact that improper handling or storage had occurred is often difficult or impossible to determine. If a shopper placed a package of meat on a shelf in another area of a store, a store employee may merely return that package to the meat case without knowing how long the meat had gone unrefrigerated. Another shopper could easily purchase that meat package, never knowing that it had been left unrefrigerated for a potentially lengthy, and unhealthy, length of time.

It would be desirable to have a system to manage and monitor the conditions of objects and individuals, as well as their environment, and to utilize the information collected to perform functions or alert a responsible party in order to reduce problems that could otherwise become severe if not for the intervention.

### SUMMARY OF THE INVENTION

It is therefore an object of the present invention to provide an apparatus and method for monitoring and detecting the status and/or change of a condition applicable to an object or to an individual. The information collected that relates to the status or change of the condition is managed to provide an appropriate response or remedy to any problems or difficulties that may be detected.

### BRIEF DESCRIPTION OF DRAWING

The above-mentioned and other advantages of the present invention, and the manner of obtaining them, will become more apparent and the invention itself will be better understood by reference to the following description of the embodiments of the invention taken in conjunction with the accompanying drawing, wherein:

FIG. 1 is a diagrammatic view of one embodiment of a condition monitoring system in accordance with the present invention.

FIG. 2 is a perspective view of a garment having a wetness sensor in accordance with one embodiment of the present invention.

FIG. 3 is an exploded perspective view of the garment depicted in FIG. 2.

FIG. 4 is a perspective diagrammatic view of a practical application of one embodiment of the present invention.

FIG. 5 is a perspective view of a sensor in accordance with one embodiment of the present invention.

FIG. 6 is a top plan view of a mattress in accordance with one embodiment of the present invention.

FIG. 7 is a diagrammatic view illustrating signal flow for a condition monitoring and management system in accordance with one embodiment of the present invention.

FIG. 8 is a diagrammatic view illustrating signal flow for the condition monitoring system shown in FIG. 7, applied to multiple locations.

FIG. 9 is a perspective view of one application in accordance with an embodiment of the present invention.

FIG. 10 is a diagrammatic view of a condition monitoring and management system in accordance with one embodiment of the present invention.

FIG. 11 is a plan view of an individual wearing sensors in accordance with an embodiment of the present invention.

FIG. 12 is a diagrammatic view illustrating multiple features and capabilities of the present invention.

FIG. 13 is an exploded diagrammatic view of another embodiment of the present invention related to monitoring of packaged food items.

Corresponding reference characters indicate corresponding parts throughout the several views.

### DETAILED DESCRIPTION OF THE PREFERRED EMBODIMENTS

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

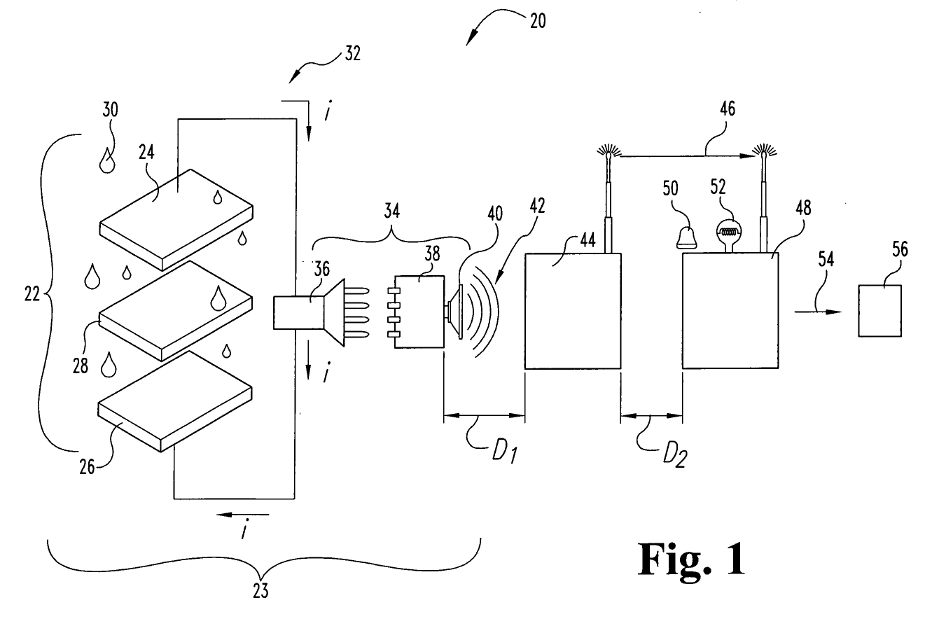

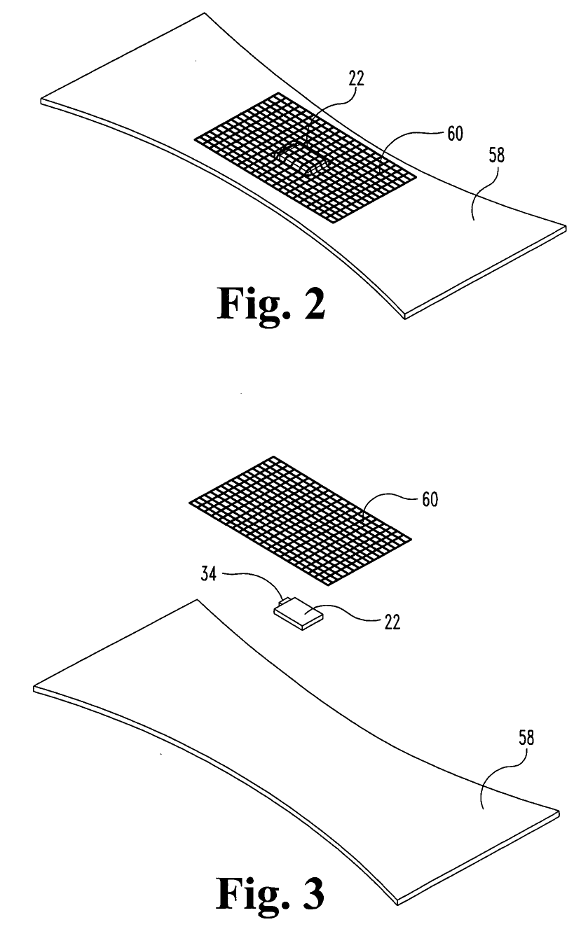

Referring now to FIG. 1, there is shown one embodiment of a system 20 of the present invention where system 20 is used for detecting and signaling that a person has become incontinent. System 20 illustratively is shown to include a wet cell battery 22, which further includes, in the example of FIG. 1, an anode 24, a cathode 26 and an absorbent material, such as membrane 28, disposed therebetween. In one application, battery 22 may be incorporated into a garment, such as a diaper, for example, as shown in FIGS. 2 and 3, and as described in more detail below. Membrane 28 is preferably impregnated with a salt, such as NaCl. In operation, when battery 22 becomes wet with urine, the salt in membrane 28 disassociates into positive and negative ions, allowing electrons to travel from the cathode back to the anode, thereby creating a current flow. In this way battery 22 acts as a wetness or incontinence detector. Wet cell batteries are well-known in the art and their operation need not be described in further detail herein. Technology is also available to manufacture paper batteries, as well as paper antennas, with conductive ink, thereby further simplifying the manufacture of the components of system 20.

In the illustrated embodiment of FIG. 1, anode 24 and cathode 26 may be made from thin foil of suitable dimension, and membrane 28 may be made from cotton cloth, cellulose fibers, non-woven polyethylene or polypropylene, or some other porous, conductive or non-conductive membrane. Anode 24 and cathode 26 may also be made from aluminum, steel, copper, composites of these and other materials suitable.

In the illustrated embodiment of FIG. 1, membrane 28 is impregnated with potassium chloride and sodium chloride in trace amounts, and the assembly of battery 22 is accomplished through a laminated assembly process.

As shown in FIG. 1, current 32 (identified by arrow i) powers annunciation device 34 that is made up of a base or first member 36 through which the current passes, and a second or attachment member 38 that incorporates a speaker 40 which produces an acoustic signal schematically illustrated by sound rings 42. In the embodiment of FIG. 1, device 34 is illustratively made in a combination of hand and automatic assembly and made available by SMT Sales, Inc., 323 Owego Street, Montour Falls, N.Y. 14865 U.S.A. Device 34 may, for example, include a semiconductor timer, such as that identified by the designation LM555 (available from National Semiconductor), which can be used to measure the degree of wetness based on the timer output frequency.

While device 34 is shown in the embodiment in FIG. 1 as producing acoustic signal 42, it would be readily recognized by one of ordinary skill in the art that current 32 generated by battery 22 could be used to power a wide variety of signal producing devices, including but not limited to, radio frequency transmitters, light bulbs, light emitting diodes

(LEDs), infrared transmitters, electromagnetic devices and the like. If device 34 is configured to produce an acoustic signal, for example, an acoustic converter 44 is preferably positioned a distance D₁ from device 34. Distance D₁ will be chosen based on the volume of the acoustic signal produced by device 34. In the illustrated embodiment of FIG. 1, converter 44 is made in a combination of hand and automatic assembly and made available by SMT Sales, Inc., 323 Owego Street, Montour Falls, N.Y. 14865 U.S.A. In one application, for example, converter 44 may be mounted to the bed of the patient wearing battery 22 and device 34. The combination of battery 22 and device 34 are referred to herein as sensor 23. Sensor 23 may be manufactured as part of a diaper and made disposable, e.g., with a paper battery and paper antenna, or it may be implemented in a reusable smart card that is inserted into a slot formed into the diaper during manufacture. The smart card could also store condition information for use at a later time.

Converter 44 receives acoustic signal 42, amplifies it and converts it to a radio signal 46 that is transmitted to transceiver 48. The distance between converter 44 and transceiver 48, shown in FIG. 1 as D₂ Clearly, D₂ can be much greater than D₁, as D₁ is limited by the signal volume or loudness of speaker 40 of device 34. In the illustrated embodiment of FIG. 1, transceiver 48 is made in a combination of hand and automatic assembly and made available by SMT Sales, Inc., 323 Owego Street, Montour Falls, N.Y. 14865 U.S.A. It should also be appreciated that system 20 may be designed and constructed to eliminate device 34, such that detection of wetness by battery 22 directly causes converter 44 to transmit a signal to transceiver 48.

In one application of the present invention, transceiver 48 may be located outside the room of a patient and mounted to a wall, for example. Transceiver 48 may be configured with one or more signal-producing devices, such as audible device 50 and/or light 52. Transceiver 48 may also relay a signal to a base station 56. In one embodiment, base station 56 is located at a health care facility nurse's station. Base station 56 may be configured to receive signals 54 from multiple transceivers, collectively designated as 48, that are located within different patient rooms in the same facility, or from different facilities entirely.

In another embodiment, base station 56 may be represented as a bracelet mounted, necklace mounted, belt mounted, or wall mounted device, any of which may incorporate a light bulb or buzzer, for example, to provide an immediate alert signal, or base 56 may contain electronic or computer memory storage for storing signals and data.

Turning now to FIGS. 2 and 3, battery 22 can be installed in a garment 58, such as a diaper, absorbent pad, sleeping garment, bed pad, wound dressing or bandage. Mesh 60 is preferably selected from a material that will spread or wick urine or some other body fluid to battery 22, thereby activating it. That is, until membrane 28 becomes wet, battery 22 does not generate a current. Many suitable materials for mesh 60 are known in the art, such as, but not limited to, polyester and natural fibrous materials. In the illustrated embodiment of FIGS. 2 and 3, garment 58 is made of multiple layers of nonwoven, spunbond polyethylene and polypropylene, laminated to multiple layers of absorbent cellulosic material. Battery 22 is inserted within the mesh layers in a combination of hand and automatic assembly and made available by SMT Sales, Inc., 323 Owego Street, Montour Falls, N.Y. 14865 U.S.A.

Conditions other than wetness alone may also be monitored. A variety of testing apparatus or components may also be incorporated within sensor 23. For example, the specific gravity of the urine may be measured to determine if the patient is dehydrated. Other measurements, such as urine pH, muscle contraction, and the amount of urine expelled, can be used to determine other health or body conditions. Other tests or measurement would be readily apparent to one skilled in the art.

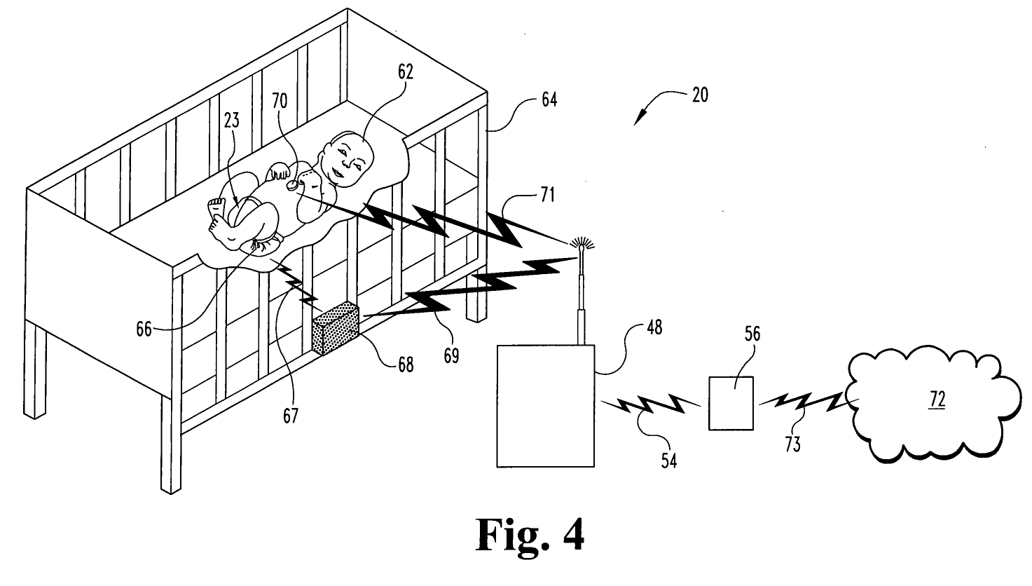

With reference to FIG. 4, the benefits of system 20 are shown. Infant 62 in crib 64 is shown wearing a garment 66 having sensor 23, as described above. Although FIG. 4 shows an infant, system 20 is equally applicable to adults as well, with a bed being substituted for crib 64. When infant 62 (or adult, as the case may be) urinates, battery 22 in sensor 23 generates a current, which in turn produces a signal 67, e.g., acoustic or otherwise. Detector 68 (which corresponds in function to converter 44 in FIG. 1) illustratively converts signal 67 received from sensor 23 to a radio frequency signal 69, amplifies it, and transmits it to transceiver 48. In turn, transceiver 48 relays a signal 54 to base 56. In the embodiment illustrated in FIG. 4, base 56 may be a central station in a day care facility, for example. Base 56 may receive multiple signals 54 from multiple transceivers 48 that are located in different rooms of the facility, for example. An operator positioned at base 56 may dispatch a caregiver to perform some service, such as changing the diaper of infant 62, when an appropriate signal 54 is received.

Other conditions of infant 62, or of the environment surrounding infant 62 can also be monitored, including blood pressure, respiration rate, body and room temperature, lighting, etc., as described in more detail below. As one example, a sensor 70 may monitor heartbeat. Sensor 70 may also be made available by SMT Sales, Inc., 323 Owego Street, Montour Falls, N.Y. 14865 U.S.A. Sensor 70 may be configured to send a signal 71 to transceiver 48 when the infant's heartbeat stops or becomes irregular. Transceiver 48 thereafter transmits a signal 54 to base 56. Advantageously, system 20 can be linked to a communications network, for example, the internet as shown by reference numeral 72. That is, information received by base 56 can be in turn transmitted to the internet, as signal 73 in FIG. 4, by conventional means, such as a computer or wireless device that may be incorporated in base 56. If infant 62 is being watched at a day-care facility, the infant's parents could log on to a specific web site to check whether the infant's diapers are timely being changed. The parents could also check other vital signs that may be monitored, such as respiration, heart rate and body temperature. These capabilities are similarly advantageous for private residences, nursing homes and other facilities. Significantly, the invention provides a powerful tool for family members or others to determine the current state of health (e.g., fever, dehydration) as well as to ensure that their loved ones are receiving proper care.

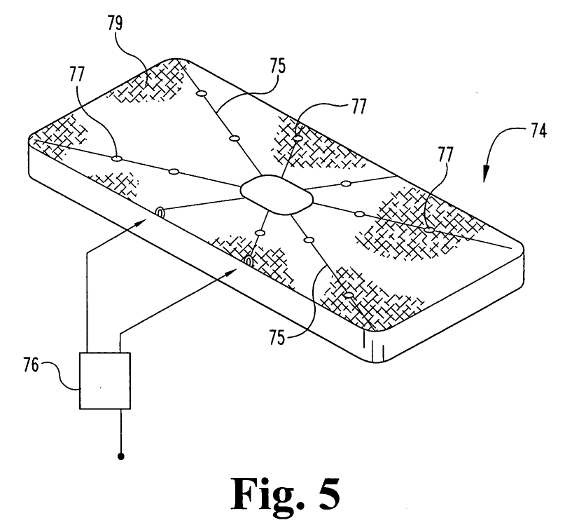

Turning to FIG. 5, a mattress 74 may be configured such that it produces a signal in response to a multiplicity of conditions, including, for example, the presence of blood, wetness due to incontinence, heart rate, respiration, body temperature, lack of movement, and the like. In the illustrated embodiment of FIG. 5, mattress 74 is a typical bed mattress that has micro-filament conductive wires 75 interspersed throughout the surface of the mattress in variable patterns and in physical contact with multiple, dispersed microsensors 77. FIG. 5 shows a cotton textile overlay 79, such as a typical fitted bed sheet, that has microfilaments 75 and sensors 77 sewn or screen printed onto textile sheet 79. Each microfilament wire 75 uses the same principal condition detection method described with respect to FIG. 1. Signals indicative of the condition detected by the microsensors are conducted via microfilaments 75 to connectors 81 that are connected to transmitter 76 in either a detachable or permanently attached manner.

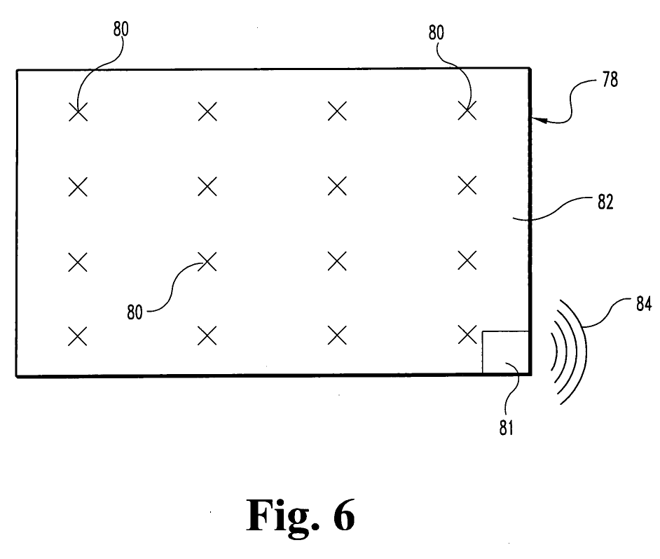

In the illustrated embodiment of FIG. 5, transmitter 76 may be employed to power the sensors 77 in mattress 74, or sensors 77 in mattress 74 may be “passive.” FIG. 6 presents another example of a mattress 78 which includes multiple transducers (i.e., sensors) 80 disposed within a “smart pad” 82 placed on top of mattress 78. Transducers 80 could be configured, for example, to detect movement, or lack thereof, in a bed. Further, transducers 80 may be configured to detect where specifically on the bed a patient is located or whether the patient is no longer in the bed. Transducers 80 may further be configured to generate a signal that is applied to annunciator 81 which in turn produces acoustic signal 84 to alert a caregiver that a particular condition has been detected. Transducers 80 may comprise a variety of different types of transducers for monitoring and detecting a variety of conditions. Annunciator 81 is therefore configured to receive signals from different types of transducers, and may be capable of determining to which condition a particular signal relates. In this way, annunciator 81 may generate different types of signals, e.g., light, bell, horn, depending upon the seriousness of the condition that is being monitored. For example, diaper wetness may cause annunciator 81 to initially turn on a light, while a sudden drop in heart rate or respiration may indicate a critically serious heath problem, such that annunciator 81 sounds a loud horn or buzzer. Annunciator 81 may also sound a buzzer or bell if a patient who has experienced diaper wetness is not attended to within a reasonable period of time following the light being lit. In this way, system 20 discriminates between signals based on their seriousness initially as well as any escalating seriousness that may result from lack of response given to earlier alert signals.

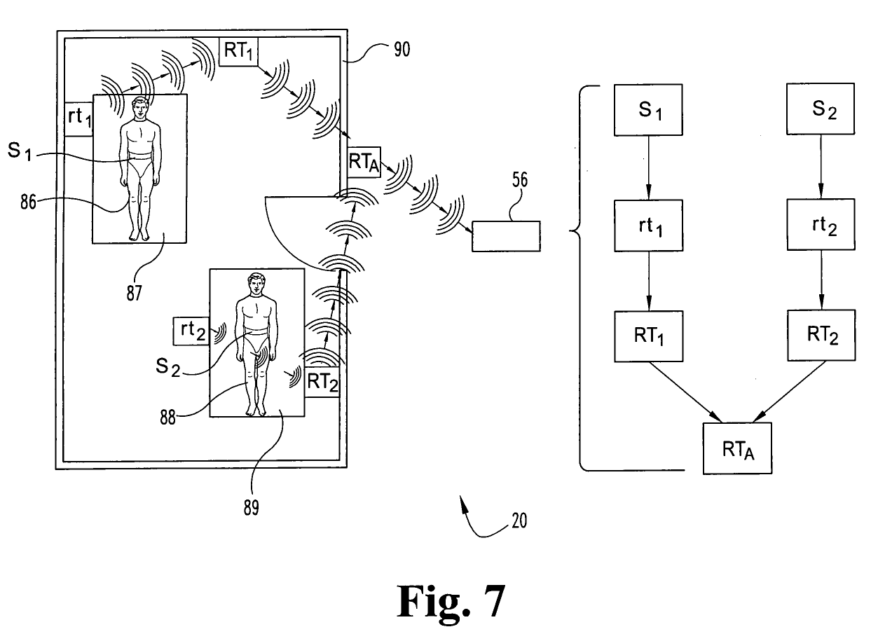

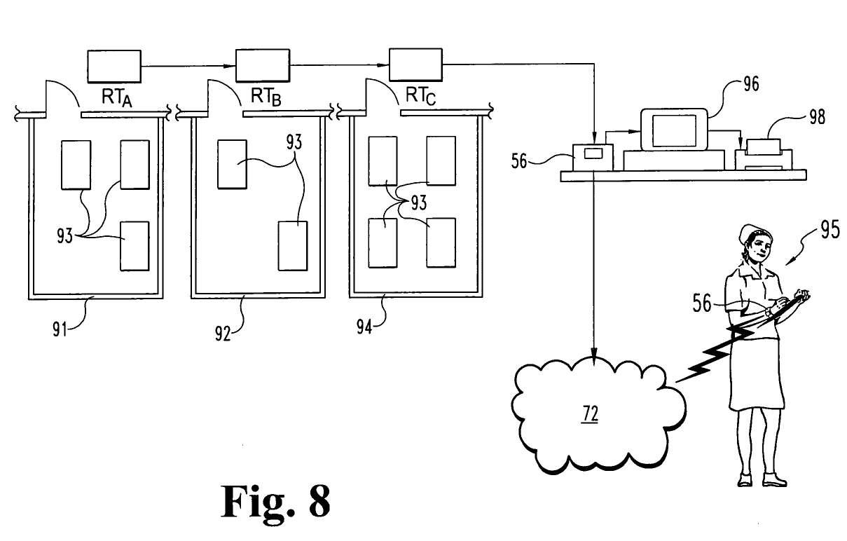

FIGS. 7 and 8 illustrate some of the capabilities of system 20 in accordance with an embodiment of the present invention. As shown, a patient 86 in bed 87 of room 90 is wearing a garment with sensor S₁ (such as sensor 23 as shown in FIG. 1). Upon occurrence of a condition (e.g., incontinence), sensor S₁ provides a signal to rt₁ (such as converter 44 as shown in FIG. 1). In turn, rt₁ provides a signal to RT₁ (which corresponds to transceiver 48 as shown in FIG. 1). Patient 88 and bed 89 are also located in room 90 and are similarly configured with sensor S₂, converter rt₂ and transceiver RT₂. An intermediate transceiver RT_{A} can be positioned outside room 90 as shown. RT_{A} can provide multiple functions, including producing a signal exterior to room 90 to alert an attendant or other passerby that there is a condition in room 90 that must be checked and attended; and transmitting data to a base station 56. Signal flow for room 90 is represented in a flowchart on the right hand side of FIG. 7. As can be seen, transceiver RT_{A} acts as an information collector or hub, and may discriminate between the signals from RT₁ and RT₂ in order to process the signal indicating the most serious or critical condition vis-à-vis patients 86 and 88.

As shown in FIG. 8, information can be relayed among several rooms 91, 92 and 94, each having one or more beds designated as 93 and configured to provide appropriate condition-identifying signals, to transceivers RT_{A}, RT_{B} and RT_{C}. Information may also be collected from each patient, or for each room, by attendant or caregiver 95 via a wrist or neck-worn base 56 as described in connection with FIG. 1. Transceivers RT_{A}, RT_{B} and RT_{C} ( . . . RTₙ) can relay their signals to base station 56 serially (as shown) or directly. Base stations 56 may relay the information to internet 72 , as shown, via a telephone or cable modem, or via an acceptable wireless protocol, such as the Bluetooth wireless protocol administered by Bluetooth SIG, Inc. Data is also received and stored by computer 96, which in turn is connected to printer 98 that may be used to generate hard copy reports.

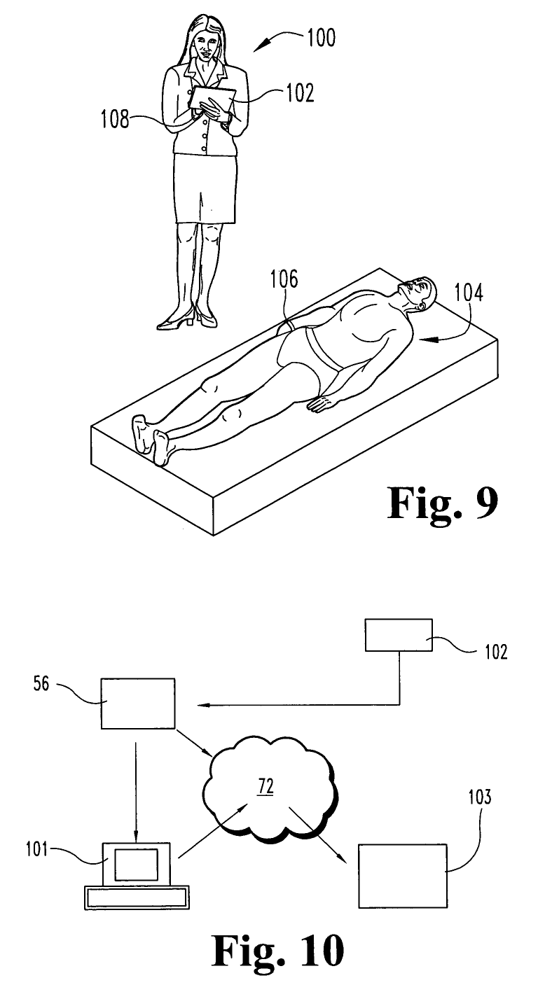

The system of the present invention can also be used to help a caregiver, physician, pharmacist or nurse to administer aid to a patient. With reference to FIG. 9, an illustrative example shows a patient 104 being treated by a nurse 100. Nurse 100 is equipped with a personal digital assistant (“PDA”) 102. One type of PDA is manufactured by 3COM and sold under the Palm brand. Other suitable PDAs are mobile phones, cellular phones and pagers. PDA 102 is configured with a recognition system that confirms nurse 100 is treating the correct patient. One such recognition system may include a fingerprint recognition pad, a heartbeat identification receiver/scanner, or some other device that can accurately verify the identify of a patient. Patient 104 may also be configured with a bracelet 106, or some other type of wearable device, which includes patient data and is capable of transmitting the same to PDA 102. Nurse 100 may also wear a bracelet 108 that includes identification information about nurse 100 that may also be transferred to PDA 102.

Once PDA 102 reads or otherwise obtains information about patient 104, either by means of bracelet 106 or by a recognition system such as those described, PDA 102 may provide instructions for nurse 100 to administer treatment such as medicine to patient 104. PDA 102 may then prompt nurse 100 to confirm that the medicine has been administered, thereby creating a patient record.

FIG. 10 illustrates how information such as that acquired from nurse 100 can be managed. As shown, patient data and treatment information stored on PDA 102 may be made available in real time on the internet, for example, to family members of the patient, or to the patient's physician or healthcare provider management or medical payor, such as a medical insurance company. As illustratively shown in FIG. 10, information from PDA 102 is transferred to base station 56, which may upload that information to internet 72 directly, or through intermediate means, such as computer 101. The information is then made available to authorized persons, as described above, via internet access device 103, such as a computer, PDA, or cellular telephone, for example. Base station may also initiate a call to emergency medical personnel, or to a patient's family, should the status of a condition warrant it. For example, if an incontinent patient had not be changed for a long period of time, base station 56 could initiate a call to the patient's spouse or other family member informing them, perhaps through a computer-generated message, that their loved one was not receiving proper care.

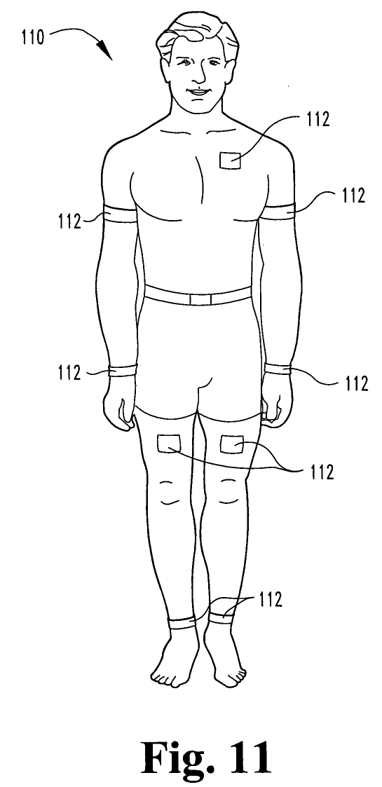

As made clear from FIG. 11, the present invention is not limited to the monitoring and management of any particular patient condition. As shown, many conditions can be monitored, such as blood pressure, brain activity, blood oxygen saturation, heart rate, respiration, incontinence, body temperature, muscle activity, impact (indicating a fall has occurred), medication history, treatment history, and patient location, or environmental conditions, such as ambient or room temperature, humidity, carbon monoxide level, to name just a few. FIG. 11 shows a representative patient 110 having a number of sensors, all designated 112, located proximal to or in contact with his body. Sensors 112 communicate with devices such as converter 44 and transceiver 48 in order to provide the necessary patient condition data which is then processed in a manner that generates appropriate alert signals in an appropriate hierarchical order indicative of the degree of criticality of the status of the condition being monitored. One application could be to use system 20 as a conventional audible baby monitor along with an indictor, such as a light, to indicate some other condition, such as wetness. In this way, a parent or caregiver hearing a baby cry over the monitor would have additional information to determine the reason for crying.

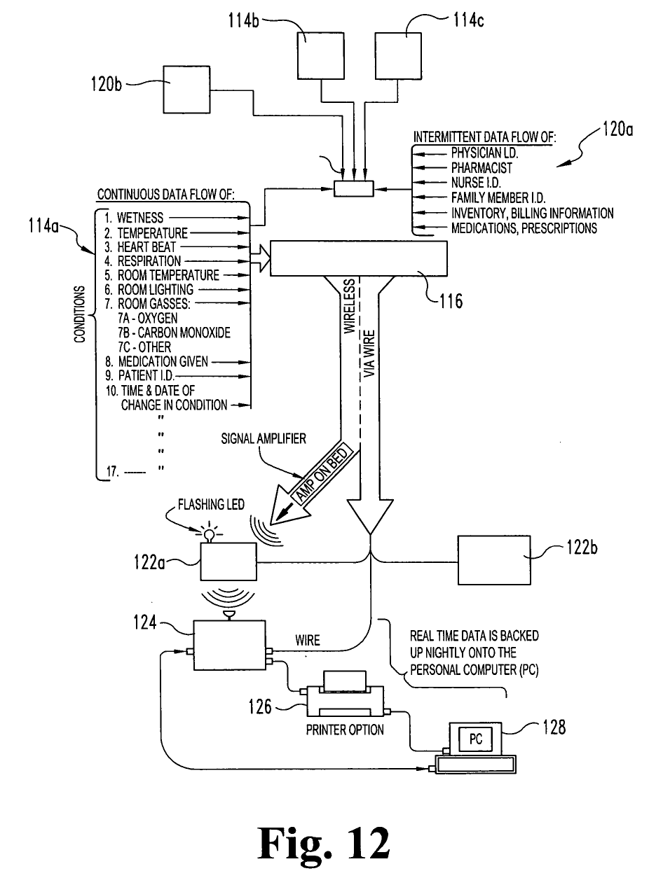

FIG. 12 provides a flowchart that illustrates through one example many of the features of the present invention and their relation to one another. The figure also illustrates data flow from the point of sensing of a condition to various data output devices. List 114a includes a number of possible conditions that may be monitored or measured by the system of the present invention. List 114a is not intended to be all inclusive, but only to act as representative examples of monitorable conditions. Typically each condition will be associated with a separate sensor, but some sensors may be sufficiently sophisticated or complex to monitor multiple conditions. Output signals from the sensors associated with list 114a are applied to processor 116, which may include devices similar to converter 44 and transceiver 48, or some other device or circuitry that performs similar functions. Other patients having their own associated lists, such as lists 114b and 114c, for example, may also provide information to processor 116. Verification of patient identity may be done by verifier 118 through fingerprint recognition or some other form of identity recognition. Caregiver or authorized person information, represented by lists 120a and 120b, may also be verified by verifier 118. Proper verification of patient and, in some cases, authorized persons, may be required by verifier 118 before processor 116 accepts information from the sensors associated with lists 114a, 114b or 114c, or those persons associated with lists 120a or 120b.

In accordance with an aspect of the present invention, processor 116 identifies and discriminates the items of information that are generated by the various sensors. The signal hierarchy, as described previously, may determine the type of alert that is activated, e.g., light, buzzer, horn, notification at nurses station, as well as any escalation of alert that may be needed due to a failure to timely respond to a lower level alert. The hierarchy may also be used to determine who has access to particular information. For example, information concerning room temperature may not be made available to the patient's family, but certainly body temperature or heart rate would most likely be made available. Processor 116 also determines the order in which signals should be processed from different patients and/or different rooms or locations. The information hierarchy created by processor 116 controls the operation of alert displays 122a and 122b. The information from processor 116 may be transmitted or otherwise transferred to various devices, such as alert devices 122a and 122b (and possibly many other alert devices as well), by either wired or wireless means. Alert device 122a is shown as also incorporating wireless transmission means, which may be via cellular phone communications, or via the internet, for example, that communicates with receiver 124. Receiver 124 may also receive information signals by hardwire means as well. Receiver 124 is illustratively shown as being connected to a printer 126 and an computer 128, although connection to other devices, by wired or wireless means, is also contemplated.

The examples given have primarily dealt with the monitoring of conditions related to infants, or to patients in health care facilities. Monitoring of individuals for other purposes is also contemplated by the present invention, such as the location of students within a school, locating a nursing home patient who wanders unnoticed out of a facility, aiding in finding abducted children or children who run away from home, or any number of other possible purposes.

Existing technology that can be useful in implementing embodiments of the present invention is available from Westinghouse lighting Systems Division under the name Retrolux. This technology incorporates wireless communications technology associated with fluorescent lighting fixtures and bulbs that may be used to provide the necessary communications link for the devices in system 20 described herein. Outdoor alert signs or billboards may also be used in connection with this technology.

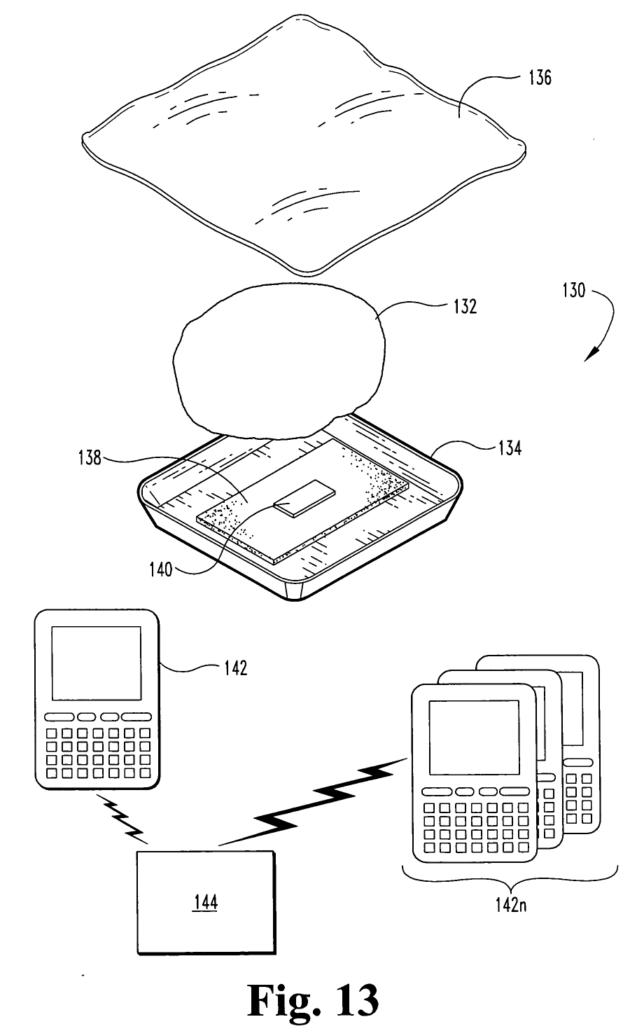

In accordance with another aspect of the present invention, condition monitoring is not limited to conditions associated with individuals. Conditions of animals or objects may be monitored as well, with physical location and body conditions of animals in homes, zoos and in the wild being desirable. FIG. 13 illustrates a monitoring system 130 which is used to monitor the condition of an object, for example, the quality of the environment in which the meat or other food is packaged. System 130 illustratively includes a selection or cut of meat 132 , e.g., steak or roast, conventionally packaged with a foam tray 134 and plastic overwrap 136. Pad 138, which may be used to control moisture within the package, is disposed between meat 132 and tray 134. A sensor 140 is placed within the package and preferably in contact with meat 132. Sensor 140 may also be incorporated within pad 138 for manufacturing purposes. Sensor 140 may be configured to monitor or measure one or more characteristics, such as, for example, current temperature, maximum temperature, moisture content, or bacterial count. Other characteristics may also be monitored as desired.

Information from sensor 140 may be collected via known, conventional means, such as by rf tagging technology, visual inspection (e.g., sensor 140 may be visible through a transparent area of tray 134) that indicates a particular condition, or other means that cause sensor 140 and any associated circuitry to transmit or otherwise transfer information to a collection device. Such a collection device might take the form of a PDA, or a wrist-worn receiver. In FIG. 13, collection device is shown as PDA 142 incorporating wireless communication technology. Information collected by PDA 142 may be transmitted to a base station 144, which may also collect information from a number of other PDAs, illustratively shown as PDA 142n. PDA 142 and/or base station 144, in accordance with an aspect of the present invention, processes the collected information and creates an information hierarchy that is used to generate alert signals that are applied to various alert systems. For example, an indication that a loaf of bread were hard or stale might generate a message to the department manager that stock should be replaced when convenient. An indication that high bacteria level were found in a package of meat could generate an alarm that required immediate action to remove that package before it was inadvertently purchased by a customer who might later become ill. Other examples of object monitoring will be apparent to one skilled in the art and are intended to be included within the scope of this disclosure.

While preferred embodiments incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A condition monitoring system comprising:
at least one monitor for providing at least one indication of at least one condition;
a detector, adapted to receive a plurality of indications from a plurality of monitors, for receiving said at least one indication and producing a signal indicative of said at least one condition and said at least one indication; and
a receiver responsive to said detector signal for generating at least one alert signal and applying said at least one alert signal to a particular one of a plurality of alert devices, said particular alert device determined by at least one of said at least one condition and said at least one indication.

2. The system described in claim 1, wherein said at least one condition comprises incontinence.

3. The system described in claim 1, wherein said at least one condition comprises body temperature.

4. The system described in claim 1, wherein said at least one condition comprises blood pressure.

5. The system described in claim 1, wherein said at least one condition comprises heart rate.

6. The system described in claim 1, wherein said at least one condition comprises respiration rate.

7. The system described in claim 1, wherein said at least one condition comprises degree of movement.

8. The system described in claim 1, wherein said at least one condition comprises physical location.

9. The system described in claim 1, wherein said at least one condition comprises ambient temperature.

10. The system described in claim 1, wherein said at least one condition comprises moisture content.

11. The system described in claim 1, wherein said at least one condition comprises a bacteria level.

12. The system described in claim 1, wherein said at least one condition comprises a maximum exposure temperature.

13. The system described in claim 1, wherein said at least one alert signal comprises an audible alarm.

14. The system described in claim 1, wherein said at least one alert signal comprises the initiation of a telephone call.

15. The system described in claim 1, wherein said at least one alert signal is accessible via the internet.

16. A method for monitoring a condition comprising the steps of:
generating at least one indication of at least one condition;
receiving said at least one indication and producing a signal indicative of said at least one condition and said at least one indication; and
generating at least one alert signal responsive to said condition indicative signal and applying said at least one alert signal to a particular one of a plurality of alert devices, said particular alert device determined by at least one of said at least one condition and said at least one indication.
